(19) United States
(12) Patent Application Publication
(10) Pub. No.: US 2007/0225610 A1
(43) Pub. Date: Sep. 27, 2007

(54) Capturing electrical signals with a catheter needle

(75) Inventors: Timothy J. Mickley, Elk River, MN (US); N. Parker Willis, Atherton, CA (US)

Correspondence Address: KENYON & KENYON LLP, 1500 K STREET N.W., SUITE 700, WASHINGTON, DC, 20005, US

(73) Assignee: Boston Scientific Scimed, Inc.

(21) Appl. No.: 11/389,060

(22) Filed: Mar. 27, 2006

(51) Int. Cl. A61B 5/04

(52) U.S. Cl. 600/509

(57) ABSTRACT

A method and medical system to take electrical readings includes an electrocardiogram (ECG) monitor, an electrode coupled to the monitor via a first lead and a needle such as an injection needle. The needle has a proximal end coupled to the monitor, where the monitor is able to measure an electrical pattern between the electrode and a distal end of the needle. In one example, the medical system is used to detect the contact, penetration, health and perforation of tissue at a target site.

### CROSS REFERENCE TO RELATED PATENT APPLICATION

The present application is related to U.S. patent application Ser. No. 11/037,154 filed on Jan. 19, 2005, which is incorporated herein by reference in its entirety.

### FIELD OF THE INVENTION

Embodiments of the present invention relate to systems and methods for the injection of therapeutic and other agents at a target site within a patient's body. More particularly, embodiments relate to the use of injection needles as electrocardiogram leads.

### BACKGROUND

Medical catheters are used for innumerable minimally invasive medical procedures. Catheters may be used, for example, for delivery of therapeutic drug doses to target tissue and/or for delivery of medical devices such as lumen-reinforcing or drug-eluting stents. Likewise, catheters may be used to guide medical instruments to a target site to perform a surgical procedure, such as tissue rescission, ablation of obstructive deposits or myocardial revascularization.

Modern catheter-based systems can be equipped with electrical sensors in order to improve the effectiveness of the catheters. In more recent approaches, these sensors can have a pair of electrodes positioned at the distal end of the catheter, where the contact surface of the catheter sensor tip typically has a planar surface area in the shape of a square, rectangle, circle, etc. The catheter sensor tip can have an opening to permit a needle or medical device to pass through the opening and into target tissue in the patient. While this approach addresses certain concerns with previous solutions, a number of challenges remain.

For example, if the tip of the catheter is not “flush” against the wall of the target tissue (e.g., heart wall tissue), the ejection of the needle may “graze” the target tissue without actually penetrating the tissue. Such could be the case even though the electrical reading indicates that the catheter tip has made contact with the target tissue. In addition, it may be difficult to determine whether the needle has penetrated to the desired depth or has penetrated all the way through the tissue and caused a perforation before injecting the therapeutic agent. Yet another difficulty relates to the fact that this approach requires the use of bulky lead wires that must run the entire length of the catheter in order to connect to the electrodes at the tip of the catheter.

### SUMMARY

One or more embodiments of the present invention are directed to improved catheter systems with sensors and related methods. In certain embodiments, a medical system includes a monitoring device, an electrode coupled to the monitoring device via a first lead and a needle having a proximal end coupled to the monitoring device, where the monitoring device measures the electrical pattern between the electrode and a distal end of the needle.

In another embodiment, the medical system includes an electrocardiogram (ECG) monitor, a standard “twelve lead” ECG configuration and another lead connected to the needle. In this regard, it should be noted that the term “lead” is sometimes used in ECG parlance to refer to a reading that is taken between two physical connections to the patient. For ease of discussion, the term “lead reading” or “electrical reading” will be used herein to distinguish readings from the physical “leads” from which they are taken. Furthermore, the term “signal” is generally used herein to refer to the electrical pattern taken from a lead, where the signal may be combined with one or more other signals to obtain a reading. Placement of the electrodes can be configured as per normal means (on the chest, arms, and legs), where lead readings can be taken by using the signals from any two of the leads—making one a “positive lead”, and the other a negative lead”. Furthermore, readings can be obtained by using any combination of the lead signals. Some lead signals can be positive and some negative, and groups of lead signals can be averaged together. Any number of leads can be used for this embodiment, as well as any position/placement for the corresponding electrodes. Electrodes could be skin electrodes, internal electrodes, or even external non-contact electrodes. It should be understood that the embodiments of this invention may use any number of leads or electrodes in any manner or any combination of electrode positions.

In another embodiment, a method of taking an electrical reading and/or tracing involves the use of a first lead attached to a skin electrode and a second lead attached to a needle. As the distal end of the needle is being guided toward heart wall tissue of the patient, the method provides for generating a tracing that indicates whether the distal end of the catheter has contacted the heart wall tissue based on the electrical reading obtained from the two leads.

Other aspects of the embodiments of the invention are set forth in the appended claims.

### BRIEF DESCRIPTION OF THE DRAWINGS

The foregoing and further features and advantages of the invention will become apparent from the following description of embodiments with reference to the accompanying drawings, wherein like numerals are used to represent like elements and wherein:

FIG. 1 is a diagram of an example of a medical system according to an embodiment of the present invention;

FIGS. 2A-2D are diagrams of examples of a sensor needle at varying stages of injection according to an embodiment of the invention;

FIG. 3 is a plot of an example of an electrocardiogram (ECG) reading according to an embodiment of the invention;

FIGS. 4A-4E are plots of examples of ECG readings from a sensor needle at varying stages of injection according to an embodiment of the invention;

FIG. 5 is a cross-sectional side view of an example of a sensor needle assembly according to an embodiment of the invention;

FIG. 6 is a flowchart of an example of a method of taking an electrical reading according to an embodiment of the invention;

FIG. 7A-7C are diagrams of examples of various reading setups according to embodiments of the invention; and

FIGS. 8A-8F are sectional views of examples of catheter tip configurations according to embodiments of the invention.

### DETAILED DESCRIPTION

Embodiments of the present invention may include a needle-based direct injection device similar to, for example, a Stiletto catheter manufactured by Boston Scientific of Natick, Mass. The tip of a needle may be used as an electrode, where the needle is connected to a monitoring device such as an electrocardiogram (ECG) monitor. The needle may be used in conjunction with standard skin electrodes to enable the monitoring of electrical signals in tissue that is in close proximity with the needle tip. For example, if the needle tip were placed at a specific location (e.g., the pulmonary veins, left ventricle or AV node of the heart), the ECG monitor may measure any distinct electrical patterns generated by the tissue. Therefore, the needle tip may be used to locate a characteristic electrical pattern known to be associated with a specific tissue location and target the location for the injection of therapeutics. The needle tip may also be used to detect the viability of contacted tissue (e.g., healthy or ischemic) and to determine whether or not the needle has penetrated and/or perforated the tissue.

It is believed that injecting certain therapeutic agents, for example, certain genetic substances, into the pulmonary veins, left ventricle and/or AV node of the heart may provide a superior treatment for certain arrhythmias, such as, bradyarrhythmia and ventricular tachyarrhythmia, and or chronic ischemia, myocardial regeneration, and myocardial remodeling. Unfortunately, certain current treatments, for example, oral drugs, radio frequency ablation, and implantable devices, lack the desired effectiveness and have undesirable side effects. Fortunately, direct injection of a therapeutic agent, for example, a gene therapy agent, into the target tissue may provide a significantly improved effectiveness and with fewer side effects.

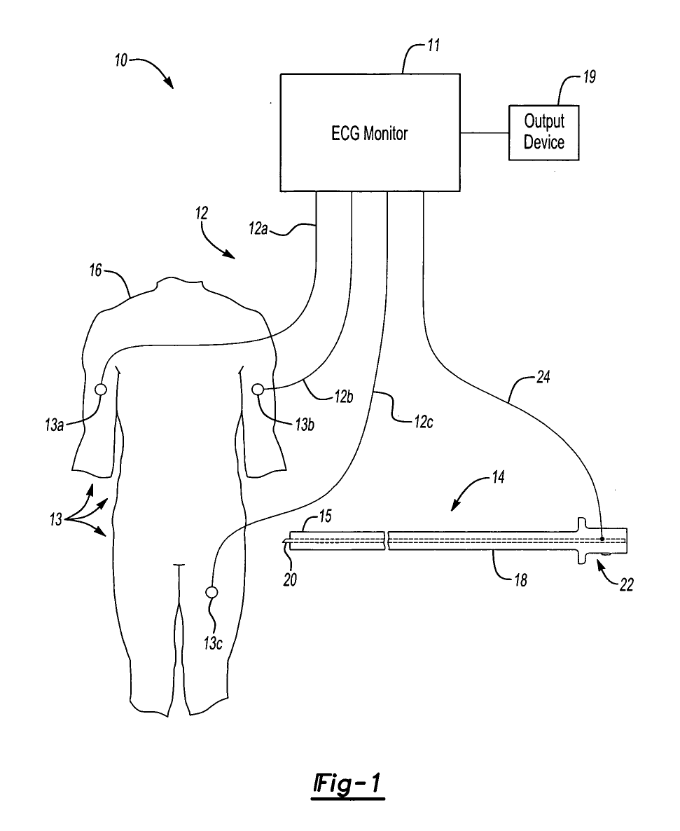

FIG. 1 is a diagram of an example of a medical system 10 according to an embodiment of the invention. Generally, the medical system 10 may monitor electrical activity of the heart and display tracings indicative of conditions such as bradyarrhythmia, tachyarrhythmia, hypertrophy, and many others. The medical system 10 may also measure tissue contact, perforation and/or penetration. In the illustrated example, an electrocardiogram (ECG) monitor (e.g., electrocardiograph) 11, is coupled to a plurality of leads 12 (12a-12c) and a needle assembly 14. The leads 12 may be attached to the patient 16 via skin electrodes 13 (13a-13c), whereas the needle assembly 14 has a needle tip 20 that may be guided toward internal tissue of the patient 16 by virtue of a catheter 18. The number of leads 12 and skin electrodes 13 can be greater or less than the number shown. For example, in one embodiment, ten skin electrodes 13 are used to take ECG measurements.

The needle is slidably disposed within the catheter 18, where the needle tip 20 is ejectable from the distal end 15 of the catheter 18. Both the electrodes 13 and the needle tip 20 can function as sensing electrodes, such that the monitor 11 is able to measure the electrical pattern (e.g., voltage and/or current) between the needle tip 20 and one or more of the other electrodes 13. In the illustrated example, the needle is coupled to the monitor 11 via a lead 24 having a first end coupled to a proximal end 22 of the needle and a second end coupled to the monitor 11. The needle assembly 14 and the lead 24 may be referred to as a “lead assembly”. The end of the lead 24 that is coupled to the monitor 11 may have a mating interface (e.g., plug) that is standard and similar to that of the leads 12. Accordingly, the illustrated needle assembly 14 is readily interchangeable with various monitors as needed.

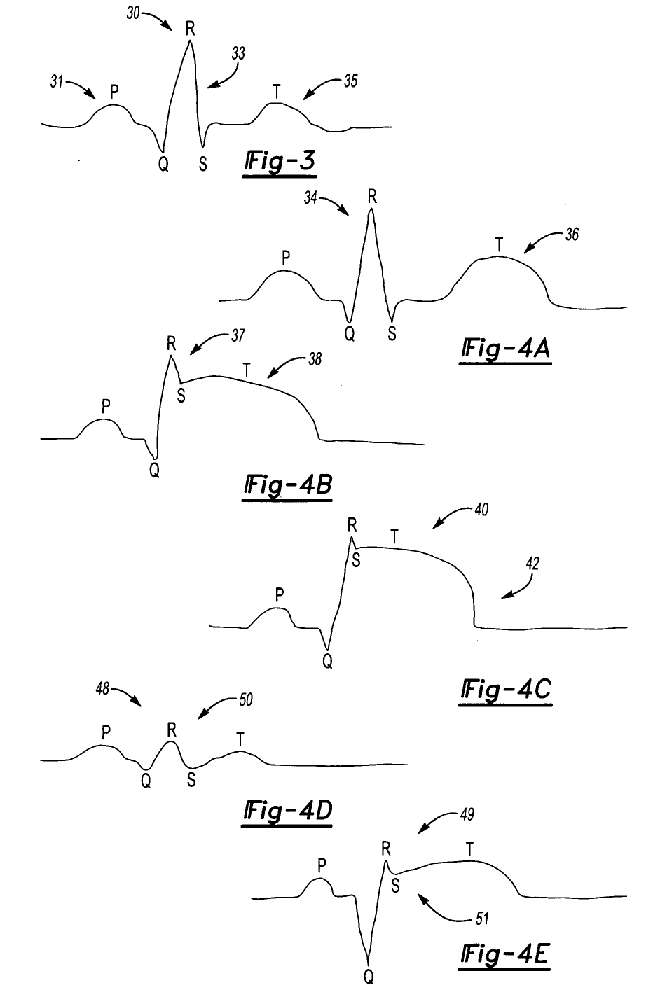

In operation, the electrodes 13 can be attached to the patient 16, and the distal end of the catheter 18 may be guided toward the target site within the patient. In one example, the catheter 18 is fed through the femoral artery in the groin area of the patient 16 toward target tissue such as heart wall tissue (e.g., myocardium) of the patient 16 in order to take ECG tracings of the patient. In this regard, FIG. 3 shows a plot 30 of an example of an ECG readout before the needle tip 20 makes contact with the heart wall tissue. In general, the plot 30 can have a P wave 31, which is the electrical signal caused by atrial contraction. The plot 30 can also have a QRS complex 33, which corresponds to the signal caused by contraction of the left and right ventricles. In particular, the Q wave, when present, represents the small horizontal (left to right) current as the action potential travels through the interventricular septum, and the R and S waves indicate contraction of the myocardium. In addition, the illustrated plot 30 has a T wave 35, where the T wave represents repolarization of the ventricles. Plot 30 depicts a normal ECG tracing of a healthy heart. Depending on the placement of leads and polarity of the leads, many different waveforms can be obtained.

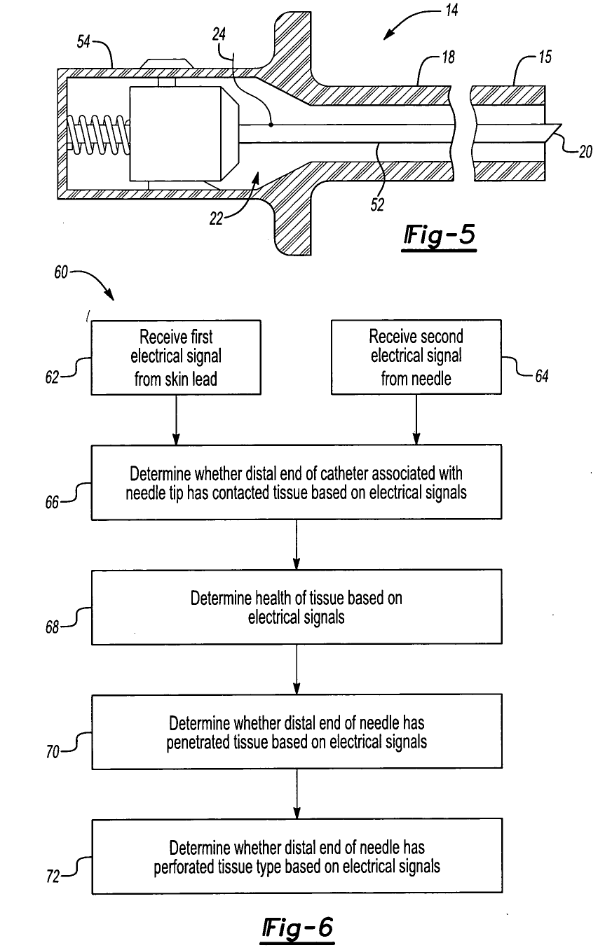

With continuing reference to FIGS. 1, 2A and 4A, the illustrated monitor 11 is able to use signals from one or more of the leads 12 and the sensor needle tip 20 to generate and/or display tracings that enable determinations to be made as to whether the catheter tip 15 has contacted a particular type of tissue such as the endocardial heart wall 32. For example, the plot 34 in FIG. 4A demonstrates that the T wave becomes more elevated and elongated resulting in a modified T wave signature 36 upon contact with the endocardium.

FIG. 4B illustrates a representative plot 37 that may be obtained as the catheter tip 15 comes into contact with the heart wall tissue and the force applied against the heart wall tissue by the catheter tip 15 increases. In this example, the ST segment becomes elevated and elongated resulting in a modified ST signature 38.

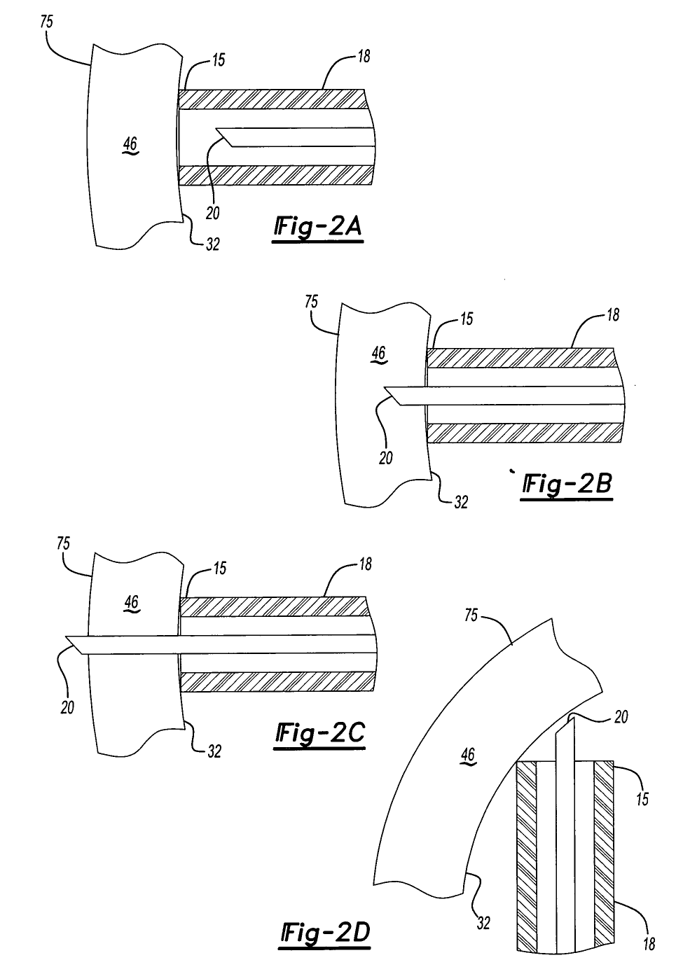

With continuing reference to FIGS. 1, 2B, 2D and 4C, the illustrated monitor 11 also is able to generate and/or display tracings that enable determinations to be made as to whether or not the needle tip 20 has penetrated into the myocardium 46 based on the electrical reading between the needle tip 20 and one or more of the electrodes 13. In particular, FIG. 2B shows the needle tip 20 engaged with the myocardium 46. The plot 40 in FIG. 4C demonstrates that the ST segment becomes even more elevated and elongated, resulting in a modified ST signature 42 upon penetration into the myocardium 46. Thus, plot 40 enables the scenario of FIG. 2B to be distinguished from that of FIG. 2D in which the needle tip 20 is positioned in the ventricle, but not engaged into the tissue 46. Such an approach provides a substantial advantage over conventional catheter-based sensors, which may be limited to the detection of tissue contact.

Turning now to FIGS. 1, 2B, 2C and 4D, the illustrated monitor 11 also is able to generate and/or display tracings that enable determinations to be made as to whether the needle tip 20 has perforated tissue such as myocardium tissue 46. In particular, FIG. 2C shows that the needle tip 20 has perforated the epicardial surface 75, as compared to FIG. 2B where the needle has only penetrated into the myocardium 46. In this example, plot 48 in FIG. 4D demonstrates that a decrease in the overall amplitude of the waveform can be exhibited, resulting in a modified waveform signature 50 upon perforation.

In yet another example, FIG. 4E shows a representative plot 49 that may be obtained as the needle tip 20 comes into contact with scar tissue. In this example, the Q wave travels lower than normal and the ST segment is slightly elevated. The result is a modified waveform signature 51.

The above signature changes are used as examples, and do not limit the scope of the embodiments of the invention. The signature waveforms and tracings described above may also have different shapes, amplitudes, polarities, etc., depending on the type, placement and number of electrodes used to obtain the tracings.

Since the distal end of the needle is in the form of a needle tip 20 having a point surface to contact the target site, the system 10 is able to detect the condition in which the needle tip 20 grazes the heart wall tissue 32 due to non-perpendicular contact. A system that uses the end of the catheter as an electrode may be unable to achieve this functionality because even though the end of the catheter has achieved contact, the needle tip itself my not be properly positioned. The system 10 may also be able to determine the viability (ischemic, healthy, scar, etcetera) of contacted tissue based on the electrical reading between the needle tip 20 and one or more of the electrodes 13. Additionally, the system 10 may be able to determine whether the needle tip 20 has passed through the myocardium tissue 46 and into the pericardial space and/or chest cavity.

The system 10 may include an output device 19 such as a display, printer, disk drive, modem, etc., that enables the electrical readings obtained between the needle 20 and the electrodes 13 to be captured and/or displayed for appropriate operating personnel such as a physician, technician, etc., to interpret the results.

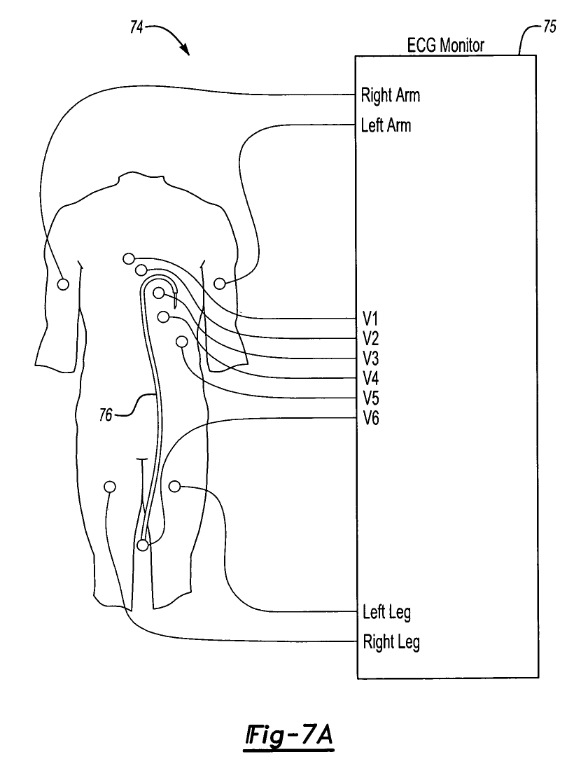

FIG. 5 shows one example of a needle assembly 14 in greater detail. In the illustrated example, the needle assembly 14 has a control assembly 54 coupled to the proximal end 22 of the needle 52, where the control assembly 54 controls extension of the needle tip 20 from the tip/distal end 15 of the catheter 18. The lead 24 can be coupled to the proximal end 22 of the needle 52 within the control assembly 54, which eliminates the need to couple the lead 24 to an electrode at the distal end of the catheter 18. As a result, the needle assembly 14 is less bulky, less expensive and easier to construct than other catheter-based assemblies. To the extent that the needle assembly 14 uses dissimilar metals, isolation of these metals from fluids such as blood or saline can be implemented to prevent galvanic reactions that may negatively affect electrical readings. In an embodiment of the present invention, the lead 24 may be of approximately 22-gauge wire, which may include a shield wire (not shown), and could be constructed of similar materials as current state of the art ECG lead wires. In an embodiment of the present invention, a protective outer covering/sheathing (not shown) may enclose the lead 24. The protective outer covering/sheathing may be, for example, a resin, a plastic and/or a heat shrink-wrap.

The needle 52 may include surfaces defining an axial passageway (not shown) that enables a fluid injection to flow from the proximal end 22 of the needle to the needle tip 20. Alternatively, a solid therapeutic agent could be fed through the needle tip 20 such that a predetermined length of the solid therapeutic agent breaks off upon injection.

As already noted, the needle assembly 14 may be used to identify a specific tissue location within a patient to deliver a therapeutic. For example, the needle assembly 14 may be located on the specific tissue location by moving the distal end 15 of catheter 18, until needle tip 20 provides for detection of a known/predetermined characteristic electrical reading for the desired specific tissue location thereby signifying contact. At this point, the needle may be actuated to extend through the opening at the distal end of the catheter 18 to enter the specific tissue location and deliver the therapeutic in exactly the desired location.

Alternate embodiments of the needle assembly 14 are also contemplated to overcome the potential loss of therapeutic at the injection site. For example, the needle may have a helical or a corkscrew-like shape that may be inserted into the specific tissue location to produce a deeper/longer needle hole, which may result in more of the therapeutic being retained in the tissue. In yet another embodiment to minimize the loss of therapeutic at the injection site, as mentioned above, the needle may deliver a solid therapeutic, for example, a polymer and cells, that may break-off in predetermined lengths when the needle is extended beyond the distal end of catheter 18 and into the target tissue.

The needle assembly 14 may also have other features such as a deflectable tip catheter, which may include a push/pull deflectable tip actuator and a lumen extending from a proximal end to a distal end of the deflectable tip actuator. A more detailed description of the operation of a deflectable tip catheter and a control assembly may be found in U.S. Pat. No. 6,083,222, issued on Jul. 4, 2000 and entitled “Deflectable Catheter for Ablating Cardiac Tissue,” which is hereby incorporated by reference in its entirety. Furthermore, more complex catheter assemblies having mechanisms such as firing distance limiting mechanisms may also be used with the needle assembly 14. A detailed description of embodiments of various catheter assemblies that may be used in embodiments of the present invention may be found in co-pending U.S. patent application Ser. No. 09/635,083, filed by the same assignee on Aug. 8, 2000 and entitled “Catheter Shaft Assembly,” which is hereby incorporated by reference in its entirety.

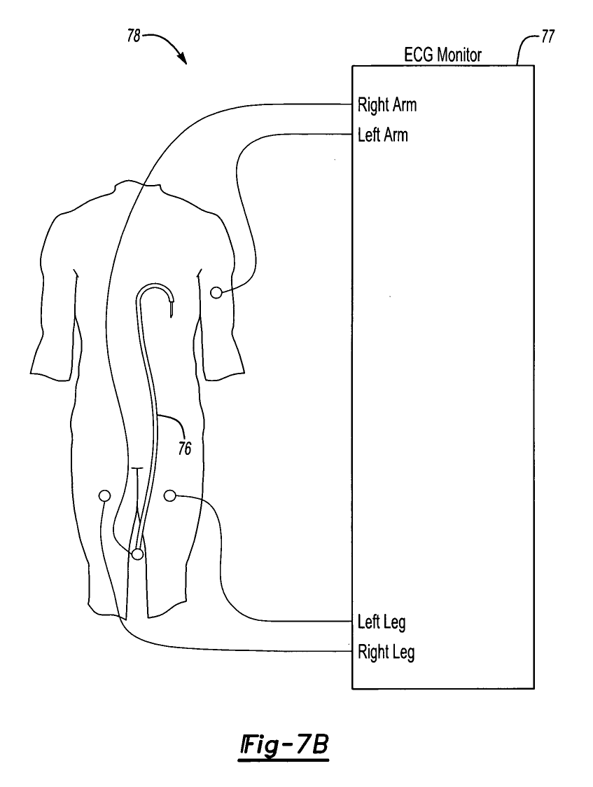

Turning now to FIG. 6, a method 60 of taking an electrical reading is shown. The illustrated method 60 may be implemented in an ECG monitor as hardware, software, firmware, and any combination thereof. For example, the method 60 may be implemented in a machine readable medium such as read only memory (ROM), random access memory (RAM), programmable ROM (PROM), flash memory, etc., as a set of instructions capable of taking electrical readings when executed by a processor. In the illustrated example, processing block 62 provides for receiving one or more first (e.g., reference) signals from one or more skin electrodes attached to a patient. A second (e.g., measurement) signal can be received from a needle at block 64, where the needle has a distal end that is being guided toward tissue such as heart wall tissue of the patient. Illustrated block 66 provides for determining whether the distal end of the catheter associated with the needle tip has contacted the heart wall tissue based on the reference signals and the measurement signal.

The health of the tissue can be determined at block 68 based on the reference and measurement signals. After the needle is extended, block 70 provides for determining whether the distal end of the needle has penetrated the tissue based on the reference and measurement signals. Block 72 provides for determining whether the distal end of the needle has perforated the tissue based on the reference and measurement signals.

Further Considerations

Two basic approaches to recording electrograms are the unipolar setup and the bipolar setup. A unipolar setup typically uses two electrodes, where one is placed near the heart and the other is placed at a far field electrical reference point, which is typically one of the limbs of the patient. A bipolar setup typically uses two electrodes as well. In this setup, however, both electrodes are placed near the heart and fairly close to each other (e.g., affixed to the same intervening device). A bipolar recording has the advantage of measuring a signal that is spatially localized to the electrodes. The closer the two electrodes are positioned to one another, the more spatially localized the signal is. This can be particularly advantageous for determining signal changes due to the proximity of the needle relative to the cardiac tissue. Bipolar recordings may present a challenge, however, because they can require two electrodes to be disposed on the same intervening device, increasing its complexity.

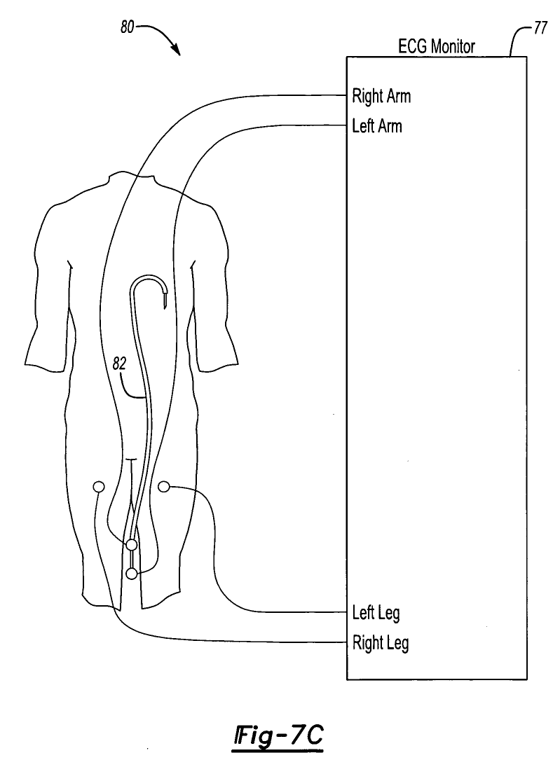

FIG. 7A shows a configuration 74 in which a full “twelve lead” setup (ten physical connections to the patient enabling twelve readings to be taken) provides for a unipolar recording from the needle of a catheter 76 to be taken by an ECG monitor 75. In the illustrated example, one of the “V” leads is attached to the catheter needle to provide a unipolar measurement signal from the needle relative to the average of three limb leads (left arm, right arm, left leg). This average of the limb leads is commonly referred to as the Wilson central terminal. The signal from the catheter needle would therefore serve as a measurement signal and the signal averaging the left arm, right arm and left leg lead signals would serve as a reference signal. The measurement signal and the reference signal can then be fed to a differential amplifier (not shown) in the monitor 75, where the output of the differential amplifier effectively represents the lead reading. In this setup, the signal from the catheter needle would therefore show up on the ECG monitor 75 as the V6 lead reading. One benefit of the illustrated setup is that all of the other ECG lead readings are preserved and available for monitoring purposes.

FIG. 7B shows a configuration 78 in which the needle of the catheter 76 can be connected to a three or four electrode ECG monitoring device 77. In this case, the other ECG signals may not be available for monitoring purposes. The lead readings taken in the configuration 78 are sometimes referred to as “Lead I” readings and “Lead II” readings, where the Lead II reading would show the unipole formed by the catheter needle relative to the left leg lead and the Lead I reading would show the unipole formed by the catheter needle relative to the left arm lead.

In FIG.7C, the configuration 80 demonstrates that the left arm lead can be attached to the needle of a catheter 82 and the right arm lead can be attached to an electrode at the distal end of the catheter 82. The signals from the right arm and left arm leads may therefore be subtracted from one another to form a Lead I reading. Therefore, in this setup the bipole formed from the two electrodes on the catheter would show up on the monitor as the Lead I reading. In addition, Lead II and Lead III readings would represent a unipolar signal of each catheter electrode relative to the left leg lead.

Bandwidth

Typically, ECG monitors have a frequency range of approximately 0.5 to 100 Hz, where some cut off as low as 50 Hz. This may be sufficient for a unipolar configuration because the signal consists of mainly low frequency far field components. Bipoles, however, can have some higher frequency content that may be suppressed by an ECG monitor. Although the signal may still be recorded with this type of equipment, the recording may not be optimal. The monitor could alternatively use a higher fidelity amplifier with a frequency range up to approximately 500 Hz in order to record a high quality bipolar signal from a device with <2 mm electrode spacing.

Electrode Material

When using the catheter needle as a recording device, care may be taken in construction of the needle and associated device. For example, if different metals are used in the construction of the device, galvanic potentials can be created that may make the recording unusable. A galvanic potential is a battery created when two dissimilar metals are exposed to an electrolytic solution and connected with an electrical conductor. There are two potential problems associated with such galvanic potentials. One is that the DC voltage can be too large for the amplifier system to which the device is connected. This can cause the amplifier in the ECG monitor to saturate, which may eliminate the signal. The other more common problem is that the potential may be unstable (e.g., vary over time), which can cause signal artifacts. These problems can be resolved by insuring that the catheter does not have dissimilar metals that are in contact with saline.

Noise artifacts can also occur if there are other metallic structures in the device that make intermittent contact with the recording electrode. This phenomenon can be worsened if two different types of metals are in contact. Noise artifacts may occur, however, even if similar metals are used. For example, noise might occur in the catheter setup if the needle is used as an electrode and is fed through a guiding catheter that has an exposed guidance coil, metal braid or other metallic structure. Such noise can be avoided by providing an insulating barrier between the needle and the guidance coil. This insulation could be applied either to the inner surface of the guide or the outer surface of the needle.

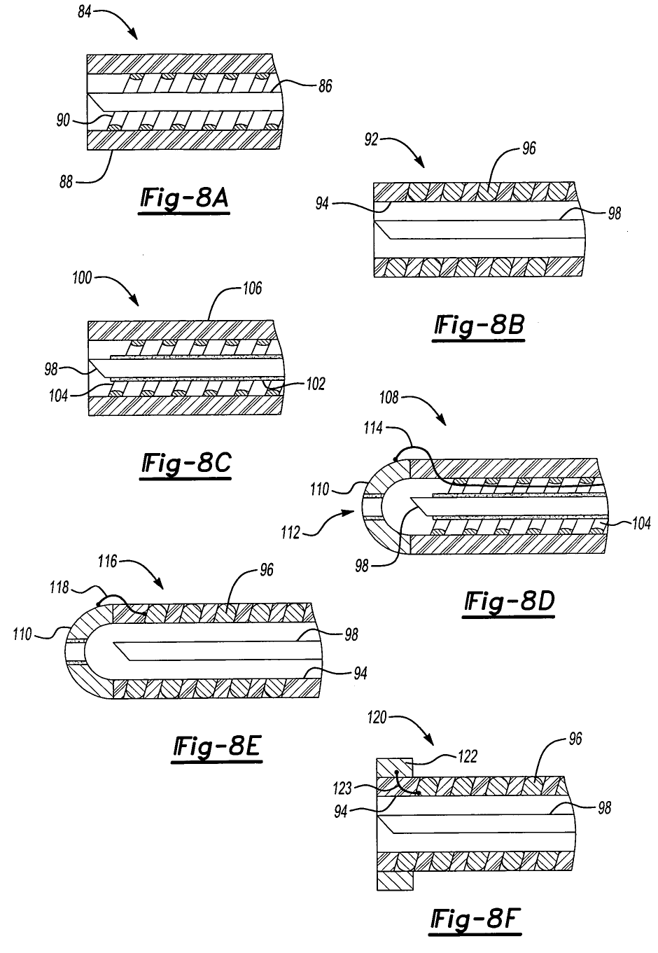

FIGS. 8A-8F show various catheter constructions to illustrate the above concepts. For example, FIG. 8A shows a catheter tip 84 having a needle 86 that is used as an electrode for obtaining electrical signals as described herein. The illustrated catheter tip 84 has an outer sheath 88 and a guidance coil 90, wherein the needle 86, sheath 88 and coil 90 are constructed of similar metals to obviate concerns related to galvanic potentials.

FIG. 8B shows a catheter tip 92 in which an electrically insulative barrier 94 is disposed between a guidance coil 96 and a needle 98. In this example, the coil 96 and the needle 98 may be constructed of dissimilar metals without concern over galvanic potentials.

Turning now to FIG. 8C, a catheter tip 100 is shown in which the needle 98 includes an electrically insulative coating coupled to the outer diameter surface of the needle 98. In this example, the guidance coil 104 and the catheter sheath 106 can be constructed of metals that are dissimilar from the metal of the needle 98 without concern over galvanic potentials. The distal end of the illustrated needle 98 does not include the insulative coating 102 in order to permit the needle to take measurements.

To further obviate concerns over noise artifacts, the electrically conductive coil and/or catheter outer sheath can be electrically coupled to ground. Such an electrical connection can be made at the proximal end of the catheter, and can significantly enhance signal quality.

FIG. 8D shows a catheter tip 108 in which a metal hood 110 at the distal end of the catheter is used as a second electrode in addition to the needle 98, which is used as an electrode as already described. The metal hood 110, which includes an opening 112 through which the needle 98 passes can be electrically coupled to the monitor (not shown) via a wire 114. The needle 98 and hood 110 can therefore be used to take bipolar signal readings. In this regard, it may be necessary to provide the monitor with a high fidelity amplifier to process the bipolar signal, as already discussed. It will also be appreciated that the interior surface of the hood 110 as well as the interior surfaces of the opening 112 can be coated with an electrically insulative material to prevent shorting between the tip of the needle 98 and the hood 110.

Turning now to FIG. 8E, a catheter tip 116 is shown in which the metal hood 110 is electrically coupled, via a wire 118, to the electrically conductive guidance coil 96, which is electrically insulated from the needle 98 by virtue of the barrier 94. The proximal end (not shown) of the coil 96 can be electrically connected to the monitor lead to complete the circuit. The illustrated example can therefore use a relatively short connection wire 118, solder joint, crimp joint, etc., which can reduce the cost, size and complexity of the overall system.

FIG. 8F shows a catheter tip 120 in which a separate electrode 122, rather than a catheter hood, is used for bipolar recordings. In this example, the electrode 122 is connected to the distal end of the guidance coil 96 via a wire 123 and the monitor lead is electrically connected to the proximal end (not shown) of the guidance coil 96. As already discussed, the electrically insulative barrier 94 prevents the electrode 122 from shorting to the needle 98.

As already noted, the sensor needles described herein can be used to deliver therapeutic agents to targeted tissue. The therapeutic agent may be any pharmaceutically acceptable agent such as a non-genetic therapeutic agent, a biomolecule, a small molecule, or cells.

Exemplary non-genetic therapeutic agents include anti-thrombogenic agents such heparin, heparin derivatives, prostaglandin (including micellar prostaglandin El), urokinase, and PPack (dextrophenylalanine proline arginine chloromethylketone); anti-proliferative agents such as enoxaprin, angiopeptin, sirolimus (rapamycin), tacrolimus, everolimus, zotarolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; bAR kinase (bARKct) inhibitors; phospholamban inhibitors; protein-bound particle drugs such as ABRAXANE™; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin−) cells including Lin-CD34−, Lin-CD34+, Lin-cKit+, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts+5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

Any of the above mentioned therapeutic agents may be incorporated into a polymeric carrier. The polymers of the polymeric carrier may be biodegradable or non-biodegradable. Non-limiting examples of suitable non-biodegradable polymers include polystrene; polyisobutylene copolymers, styrene-isobutylene block copolymers such as styrene-isobutylene-styrene tri-block copolymers (SIBS) and other block copolymers such as styrene-ethylene/butylene-styrene (SEBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

A polymeric carrier used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent/therapeutic agent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The mixture may comprise multiple polymers and/or multiple therapeutic agents.

The medical device may contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

Although embodiments of the present invention have been disclosed in detail, it should be understood that various changes, substitutions, and alterations may be made herein, and the present invention is intended to cover various modifications and equivalent arrangements. Other examples are readily ascertainable from the above description by one skilled in the art and may be made without departing from the spirit and scope of the present invention as defined by the following claims.

The term “coupled” is used herein to refer to any connection, direct or indirect, and unless otherwise stated may include a mechanical, electrical, optical, electromagnetic, integral, separate, or other relationship between the components in question. Furthermore, any use of terms such as “first” and “second” do not necessarily infer a chronological relationship.

Although embodiments of the present invention have been disclosed in detail, it should be understood that various changes, substitutions, and alterations may be made herein, and the present invention is intended to cover various modifications and equivalent arrangements. Other examples are readily ascertainable from the above description by one skilled in the art and may be made without departing from the spirit and scope of the present invention as defined by the following claims.

What is claimed is:

1. A medical system comprising:
a monitoring device;
an electrode coupled to the monitoring device via a first lead; and
a needle having a proximal end coupled to the monitoring device, wherein the monitoring device measures an electrical pattern between the electrode and a distal end of the needle.

2. The system of claim 1, wherein the needle is coupled to the monitoring device via a second lead having a first end coupled to the proximal end of the needle and a second end coupled to the monitoring device.

3. The system of claim 1, wherein the monitoring device is configured to generate a tracing that indicates whether a catheter tip associated with the distal end of the needle has contacted tissue based on the electrical pattern.

4. The system of claim 3, wherein the monitoring device is configured to generate a tracing that indicates whether the distal end of the needle has penetrated the tissue based on the electrical pattern.

5. The system of claim 4, wherein the monitoring device is configured to generate a tracing that indicates whether the distal end of the needle has perforated the tissue based on the electrical pattern.

6. The system of claim 3, wherein the tissue comprises myocardium tissue.

7. The system of claim 3, wherein the monitoring device further is configured to generate a tracing that indicates a health of the tissue based on the electrical pattern.

8. The system of claim 3, further including an output device to display the tracing.

9. The system of claim 1, wherein the electrical pattern includes a unipolar signal, the electrode includes a skin electrode and the monitoring device includes an electrocardiogram (ECG) monitor.

10. The system of claim 9, further including a plurality of skin electrodes, wherein each skin electrode is coupled to the monitoring device via a corresponding lead and the ECG monitor measures an electrical pattern between the distal end of the needle and one or more of the plurality of skin electrodes.

11. The system of claim 1, wherein the electrical pattern includes a bipolar signal and the monitoring device includes an electrocardiogram (ECG) monitor having a high fidelity amplifier to process the bipolar signal, the system further including a catheter having the first electrode disposed at a distal end of the catheter, the needle being slidably disposed within the catheter and the distal end of the needle being ejectable from the distal end of the catheter.

12. The system of claim 11, wherein the electrode includes an electrically conductive hood.

13. The system of claim 11, wherein the catheter further includes an electrically conductive guidance coil and the electrode is coupled to the lead via the coil.

14. The system of claim 13, wherein the needle and the coil are comprised of similar metals.

15. The system of claim 13, wherein the needle includes an electrically insulative coating coupled to an outer diameter surface of the needle.

16. The system of claim 13, wherein the catheter includes an electrically insulative barrier disposed between the coil and the needle.

17. An electrocardiogram (ECG) lead assembly comprising:
a needle having a proximal end and a distal end; and
a lead having a first end coupled to the proximal end of the needle.

18. The lead assembly of claim 17, wherein the needle and the lead are adapted to transport an electrical signal between the distal end of the needle and a second end of the lead.

19. The lead assembly of claim 18, further including:
a catheter having a proximal end and a distal end, the needle being slidably disposed within the catheter; and
a control assembly coupled to the proximal end of the needle, the control assembly to control extension of the distal end of the needle from the distal end of the catheter.

20. The lead assembly of claim 19, wherein the catheter includes an electrode disposed at the distal end of the catheter.

21. The lead assembly of claim 20, wherein the catheter further includes an electrically conductive coil and the electrode is coupled to the coil.

22. The lead assembly of claim 19, wherein the first end of the lead is coupled to the proximal end of the needle within the control assembly.

23. The lead assembly of claim 19, wherein the distal end of the needle is in the form of a needle tip having a point surface to contact a target site.

24. The lead assembly of claim 17, wherein the needle includes an axial passageway to enable a fluid injection to flow from the proximal end of the needle to the distal end of the needle.

25. The lead assembly of claim 17, wherein the needle is adapted to deliver a solid therapeutic agent, the needle to break off a predetermined length of the solid therapeutic agent.

26. A method comprising:
receiving a reference signal from a skin electrode attached to a patient;
receiving a measurement signal from a needle having a distal end that is being guided toward heart wall tissue of the patient; and
generating a tracing that indicates whether a catheter tip associated with the distal end of the needle has contacted the heart wall tissue based on the reference signal and the measurement signal.

27. The method of claim 26, further including generating a tracing that indicates whether the distal end of the needle has penetrated myocardium tissue of the patient based on the reference signal and the measurement signal.

28. The method of claim 27, further including generating a tracing that indicates whether the distal end of the needle has perforated the myocardium tissue based on the reference signal and the measurement signal.

29. The method of claim 27, further including generating a tracing that indicates a health of the myocardium tissue based on the reference signal and the measurement signal.

30. The method of claim 26, further including:
receiving a plurality of reference signals from a plurality of skin electrodes; and
generating a tracing that indicates whether the catheter tip associated with the distal end of the needle has contacted the heart wall tissue based on one or more of the plurality of reference signals and the measurement signal.
